(19) United States
(12) Patent Application Publication
(10) Pub. No.: US 2021/0228084 A1
(43) Pub. Date: Jul. 29, 2021

(54) LED INDUCED FLUORESCENCE DETECTION SYSTEM OF EPITHELIAL TISSUE

(71) Applicant: Fortis BioPharma LLC, Magnolia, TX (US)

(72) Inventor: Cooper Collins, Magnolia, TX (US)

(73) Assignee: Fortis BioPharma LLC, Magnolia, TX (US)

(21) Appl. No.: 17/158,315

(22) Filed: Jan. 26, 2021

Related U.S. Application Data
(60) Provisional application No. 62/966,200, filed on Jan. 27, 2020.

(51) Int. Cl. A61B 5/00; G02B 5/20

(52) U.S. Cl. CPC A61B 5/0071; G02B 5/208; A61B 5/444; A61B 5/742; A61B 2560/0214; A61B 2562/0233; A61B 2562/185; A61B 2560/0425; A61B 2560/0431; A61B 5/0077

(57) ABSTRACT

A device for irradiating epithelial tissue with a light source between 410 nm and 420 nm and observing the presence of resultant fluorescence induced in the tissue. The device may include a long pass filter blocking light reflected from the tissue surface of 500 nm or shorter. A method for detecting epithelial neoplasia by irradiating tissue with a light source between 410 nm and 420 nm and observing the presence resultant fluorescence induced in the tissue. The method may include blocking reflected light having a wavelength of approximately 500 nm or shorter. The device may be a handheld device containing a light emission source and viewport with long pass filter. The light emission source can be positioned substantially normal to the tissue surface. The device may include a camera and a separately located display and recording capability. The light emission source can be positioned on a handle or upon the periphery of a viewport.

### RELATED APPLICATION

This disclosure claims priority to provisional application Ser. No. 62/966,200 entitled “LED Induced Fluorescent Detection System of Epithelial Tissue” filed Jan. 27, 2020 and which is incorporated by reference herein in its entirety.

### FIELD OF USE

This disclosure pertains to device using an LED light emission source emitting a specified narrow range of light wavelength. The center wavelength may preferably be between 410 nm and 420 nm and with an average of 415 nm. The light may be directed external to the skin or subcutaneously to living tissue. The disclosure detects the presence of fluorescence emanating from the tissue in response to the light emission source. This detected fluorescence may be indicative of the health of the tissue.

### PRIOR ART

Devices utilizing light to induce fluorescence in tissue are known. However, such devices have utilized light sources whose output is poorly matched to the fluorescent light output band from the fluorescing tissues they are intended to excite. Such devices have typically used light sources whose visible emission bands overlap the fluorescence band of the excited tissues, thereby requiring high pass filtration at the light source to avoid overlap.

### BRIEF SUMMARY OF DISCLOSURE

In 2019, approximately 1,800,000 people will be diagnosed with cancer. The majority of these cancers will be of epithelial origin, i.e., relating to or denoting the thin tissue forming the outer layer of a body's surface and lining the alimentary canal and other hollow structures) and stroma (supportive tissue). Such tissues may be skin, oral, cervical and colorectal. Epithelial tissues are widespread throughout the body. They form the covering of all body surfaces, line body cavities and hollow organs, and are the major tissue in glands. They perform a variety of functions that include protection, secretion, absorption, excretion, filtration, diffusion, and sensory reception.

Early detection of pre-invasive epithelial neoplasia has the potential to increase patient survival and improve quality of life. Neoplasia is abnormal cell growth. It can result in tumors which may be benign or cancerous. Many of the currently available screening and detection techniques for epithelial pre-cancers do not provide adequate sensitivity and specificity; furthermore, many screening and detection methods require extensive training to yield adequate clinical results. Pre-invasive epithelial neoplasia preinvasive cancer may be a cluster of malignant cells that has not yet invaded the deeper epithelial tissue or spread to other parts of the body, i.e., carcinoma in situ.

For example, to discern between premalignant and early malignant lesions from common benign inflammation in suspected oral cancer cases, practitioners commonly perform visual examinations. However, visual screenings have been reported to have a sensitivity of only 74%, a specificity of 99%, and a negative predicative value of 0.67 and 0.99, respectively. Consequently, practitioners often resort to the invasive and painful option of biopsies to confirm the presence of precancer or even early cancer. Thus, despite the easy accessibility of the oral cavity for examination, current methods do not adequately screen and detect precancers in a non-invasive manner.

There is a need for a non-invasive tool to diagnose epithelial neoplasia, such as oral cancer, skin cancer, cervical cancer and colon cancer that yields accurate results. The Applicant's disclosure provides a simplified and low cost device and method for diagnosing epithelial neoplasia. One application of the device subject of this disclosure facilitates the examination of skin tissue.

There are a range of light-tissue interactions that can be exploited to improve the visualization of neoplastic lesions. In particular, tissue autofluorescence has recently shown promise as an adjunctive diagnostic tool. Fluorophores within the oral epithelium (relating to or denoting the thin tissue forming the outer layer of a body's surface and lining the alimentary canal and other hollow structures) and stroma (supportive tissue) absorb UV and visible light and can re-emit some of this light at longer wavelengths in the form of fluorescence. Prior art methods require blocking the reflected illumination light with an absorbing filter such as an expensive dichroic filter. This blocking has been required to prevent interference with detection of subtle or weak fluorescence signals. The present disclosure eliminates the need of such blocking filter by restricting the wavelength of the light emission source.

With the Applicant's device, it is possible to visualize the longer wavelength fluorescence even with the naked eye. Autofluorescence originates from a variety of fluorophores in the epithelial structure and is sensitive to alterations in both tissue morphology and biochemistry associated with neoplasia. In one embodiment of the Applicant's disclosure, the device can detect oral cancer and precancer through the detection of a loss of autofluorescence across a broad range of UV and visible excitation wavelengths. This loss of fluorescence is largely attributed to a decrease in fluorescent crosslinks associated with stromal collagen that underlies the neoplastic lesion.

This disclosure teaches using a better-matched LED emitter and superior low pass viewing filter, thereby eliminating the need for a high pass filter at the light emission source. Further the disclosure eliminates emissions that pass beyond the cutoff of the low pass filter, e.g. wavelengths longer than 460 nm. Therefore, the tissue surface is only illuminated by light having wavelengths less than approximately 460 nm. Also subject of the disclosure includes a beam-shaping collimating optic affixed to the LED. Further the disclosure teaches a charging port. The disclosure also teaches variable positional light emission source or viewing filter. Other advantages are disclosed and will be appreciated by persons skilled in the art.

### BRIEF DESCRIPTION OF DRAWINGS

The accompanying drawings, which are incorporated in and constitute a part of the specification, illustrate preferred embodiments of the disclosure. These drawings, together with the general description of the disclosure given above and the detailed description of the preferred embodiments given below, serve to explain the principles of the disclosure. The drawings do not limit or exclude other embodiments of the disclosure.

FIG. 1 illustrates an example of the wave band of the emitted LED light utilized by the Applicant. The illustrated wavelength center is approximately 405 nm.

FIG. 2 is a perspective view of one embodiment of the Applicant's device. Illustrated is a self contained device wherein electrical power, light emitter and collimator is contained in a handle attached to a viewing detector or viewing filter. The illustrated circular viewing detector comprises a translucent long pass light filter, e.g., blocks wavelengths less than 460 nm.

FIG. 3 is a cross sectional view of one embodiment of the Applicant's device as illustrated in FIG. 2.

FIG. 4 is an expanded side view of showing the collimator, heat sink, and LED emitter of the embodiment illustrated in FIG. 3. Also illustrated is a compressible component that can activate a switch for the LED emitter.

FIG. 5 illustrates a side view of the device in relation to illuminated and fluorescent tissue subject of examination. The viewport filter, i.e., fluorescent light detector, can be mounted in a single device with the collimator, heat sink, LED emitter, switch and battery. The latter components may comprise a hand holdable handle affixed to the viewport filter as shown in FIGS. 2 and 3.

FIG. 6 illustrates the wavelength filtering of the long pass filter utilized in the fluorescence light detector (viewing filter). In the illustrated example, light with wavelengths greater than 460 nm pass through the filter and are therefore visible to the user.

FIG. 7 illustrates a side view of an alternate embodiment of the disclosure showing the LED emitter and collimator orientation (emission axis) variable with a rotation component located on the handle.

FIG. 8 illustrates a side cross sectional view of another alternate embodiment of the disclosure showing the viewport filter, i.e., fluorescent light detector, pivotably mounted on the device handle wherein the axis of light reception varies with the fixed axis of LED light emission through the collimator.

FIG. 9 is a perspective illustration of another embodiment of the disclosure wherein the collimator (optic) is positioned adjacent to the viewport filter. It will be understood that the LED emitter is located beneath the visible collimator. Also shown is a camera positioned beneath the viewport filter and receiving the induced fluorescence.

FIG. 10 is a perspective illustration of another embodiment of the disclosure wherein a camera is positioned in the frame of the viewport filter.

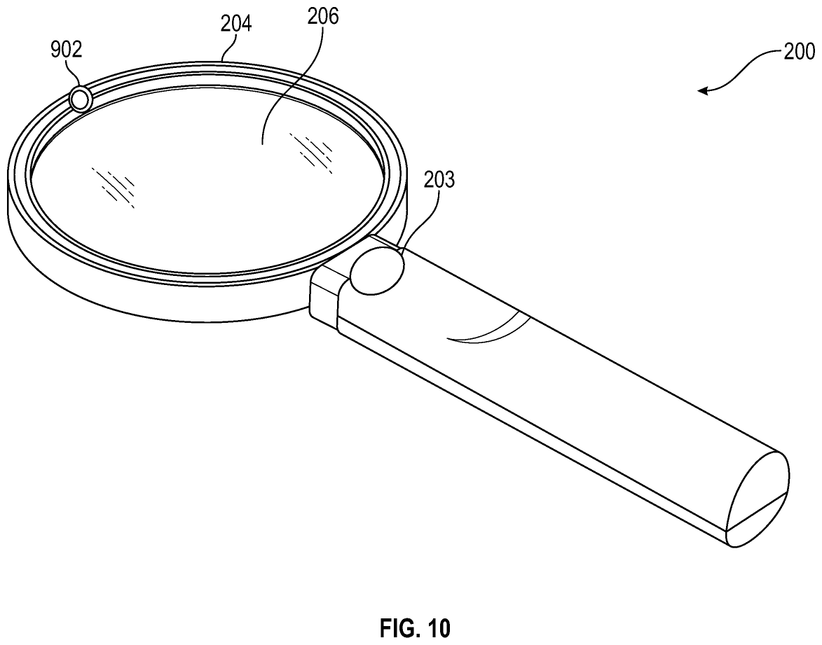

FIG. 11 is a perspective view of the device of the disclosure wherein multiple collimators and underlying LED emitters are positioned on the outer border or frame of the viewport filter.

FIG. 12 is another perspective view of the device of the disclosure illustrating the combination collimator and LED emitter positioned adjacent to the viewport filter.

FIG. 13 is a schematic illustration of the device utilizing a camera attached to a separately located image display component.

### DETAILED DESCRIPTION OF THE DISCLOSURE

Noninvasive and accurate techniques that facilitate the early detection of neoplastic changes improve cancer survival rates and lower treatment costs by reducing or eliminating other diagnostic procedures and allowing prompt commencement of treatment. Optical tools using knowledge of light and tissue interaction have been developed for noninvasive methods of cancer diagnosis. These tools have utilized broad ranges of light and required high frequency light band pass filter.

This disclosure utilizes a selected light emission source to induce fluorescence in living tissue, e.g., epithelial tissue. The selected light source features an output band whose higher frequency avoids overlap with the induced fluorescence output band of the target tissue. As will be explained in greater detail below, the collagen matrix present in healthy tissue fluoresces when exposed to a light source.

Normally, reflected white light on objects is observable because of a dominant light-tissue interaction. However, the teaching of this disclosure illustrates a device and method improving and facilitating the observance of tissue autofluorescence, in which optical contrast between normal and neoplastic tissue might be significantly greater. The disclosure teaches that by selection of light emitters, it is therefore not necessary to employ high pass filtration (high frequency/short wavelength) in front of the emitter to limit emitter bandwidth on the low frequency end of the band so as to avoid overlap with the induced fluorescence band. As discussed herein, the excited or induced fluorescent light from the epithelial tissue is of a low frequency, long wavelength. Neoplastic tissue is a new, often uncontrolled growth of abnormal tissue or tumor. In some cases, neoplastic tissue is cancerous or pre-cancerous.

When molecules within epithelial tissue absorb incident light, they can release energy in the form of fluorescent light. The intensity and color of the fluorescence gives information about the local biochemical composition of tissue. Molecules capable of emitting light caused by optical excitation are called fluorophores. Autofluorescence originates from many endogenous fluorophores present in the tissue such as the crosslinks in the structural proteins collagen and elastin, the metabolic co-factors nicotinamide adenine dinucleotide (NADH) and flavin adenine dinucleotide (FAD+), aromatic amino acids, such as tryptophan, tyrosine, and phenylalanine, and porphyrins. Tissue fluorescence signatures are of particular interest, because spectral changes might reflect changes in metabolic activity, e.g., neoplasia, and communication between the epithelium and the stroma.

Noninvasive and accurate techniques that facilitate the early detection of neoplastic changes improve survival rates and lower treatment costs by reducing or eliminating other diagnostic procedures. Optical tools using knowledge of light and tissue interaction can provide fast, noninvasive methods of cancer diagnosis. Normally, reflected white light on objects is observable because of a dominant light-tissue interaction. However, it is also possible to observe tissue autofluorescence, in which optical contrast between normal and neoplastic tissue might be significantly greater.

As such, embodiments of the present disclosure provide a convenient, simple, low cost and hand held autofluorescence imaging system and method in which the illumination and observation conditions have been optimized to take into account the limitations of the human visual system.

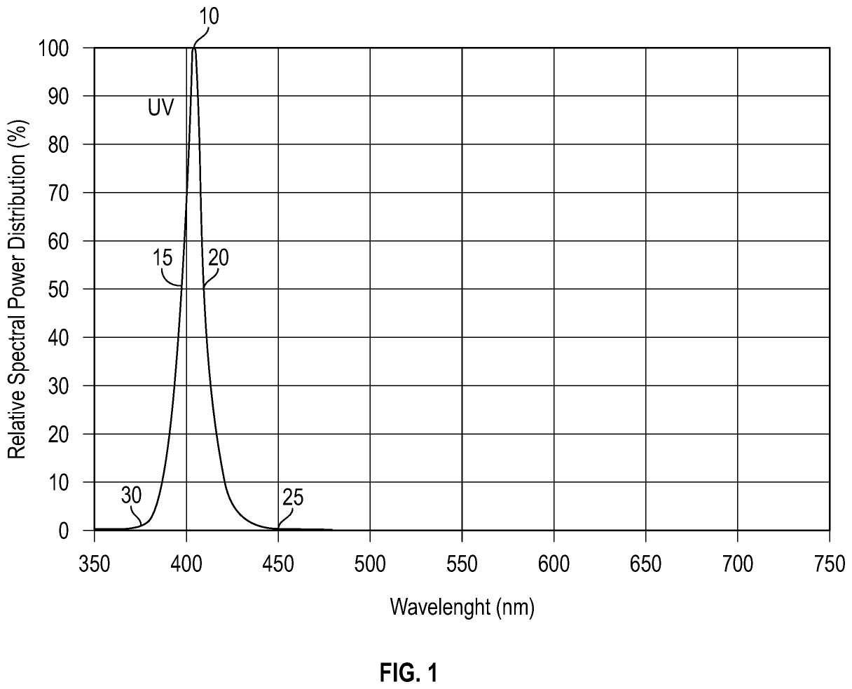

This disclosure provides an improved method and device. The disclosure utilizes an LED emitter to excite the subject tissue. Referencing FIG. 1, the light from the LED emitter has a narrow band width. The light wave has a peak or center 10 at 405 nm. In other embodiments, the light peak may be between 400 nm and 420 nm. It will be appreciated that the LED emitter is selected to have a wavelength distinct from the wavelength of fluorescent light induced in the tissue.

The breadth of the wave length is often described by the wave length value at 50% of the amplitude of the peak, 15, 20. For the emitter wavelength illustrated in FIG. 1, the values are approximately 398 nm and 410 nm. This may be sometimes referred to as the “half maximum”.

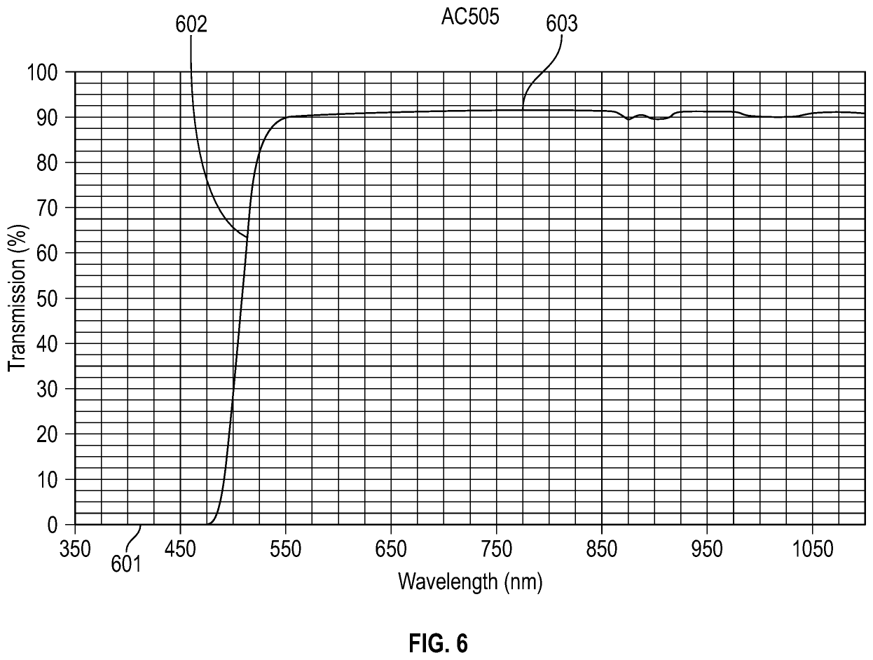

For the Applicant's disclosure, the center wavelength may preferably be between 410 nm and 420 nm and with an average of 415 nm. For the peak of 415 nm, the half maximum will be 410 nm and 420 nm respectively. The 0% range may be 50 nm from the peak wave length. Note this will be well below the range of light wavelength 602, 603 passing through the long pass filter, reference FIG. 6. In the example FIG. 1, the 0% point 25, 30 is 450 nm and 380 nm respectively.

This shorter wavelength emitted from the LED light of the disclosure eliminates the need for a high band pass filter. It will be appreciated that such a filter blocks the longer wave lengths (such as wavelengths comparable to the induced fluorescent light). Therefore, long wave lengths from the emitted light do not interfere with the induced fluorescent light. Such a high band pass filter is required by the prior art devices to be placed in front of the device light source. In one prior art embodiment, a costly Woods Glass (dichroic high pass) filter is employed. It will be appreciated that such filter decreases efficiency, i.e., reduction of overall light output by 20-40%, in addition to added cost and weight to the device, as well as problems of complexity, cleaning, maintenance and calibration.

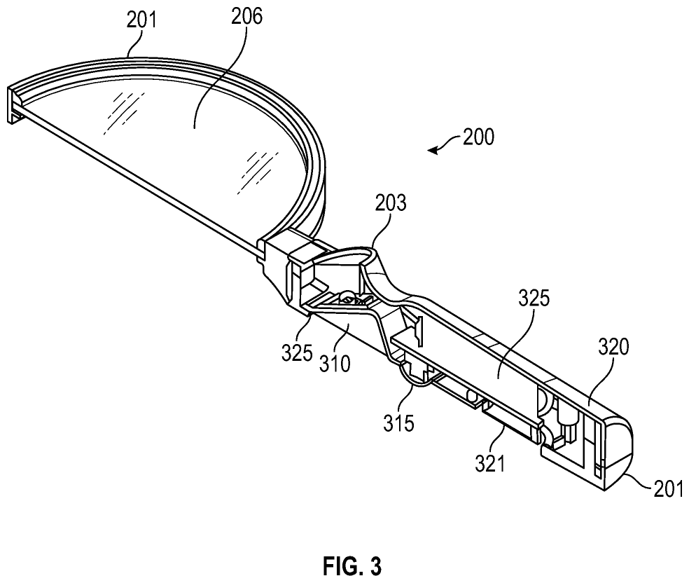

This disclosure also may be utilized with a single emitter and reducing the number of emitters required. See for example FIG. 3 and violet light source (relatively short wavelength) 325. Specifically, by using an emitter whose center wavelength is 410-420 nm, the Applicant's disclosure eliminates light emissions that pass beyond the cutoff of the long pass filter as discussed further herein. (Reference FIG. 6 602.) It will be appreciated that the prior art devices employ light sources emitting between 300 and 650 nm. In some examples of the prior art, the emitter wave length is centered at approximately 445 nm, with the low frequency portion of the band extending well into the fluorescence band of the target tissues, thereby necessitating use of a high pass filter to block such portion of the emitter's output band. The range of fluorescence of the tissue induced by the light transmitter is 500 to 600 nm.

It will be appreciated that the Applicant's disclosure allows performance with a single LED and one larger-diameter collimator. A collimator is a device that produces parallel lines of rays of radiation. The single LED and one larger-diameter collimator has enhanced performance to prior art devices that may utilize three LEDs and three smaller collimators in the same footprint. Not only is there significantly less light loss, but the attainable level of collimation keeps more of the beam within the desired viewing area, resulting in the single LED performing equivalently to the three-LED configuration. Part of this is due to the loss of over 20% of the light output as a result of the 450 nm emitters employed in the other (prior art) design having to be passed through a dichroic high pass filter, but the balance is due to the use of an appropriately-sized optic (collimator) 203 relative to the emitter size. Because optical efficiency of the collimator decreases as collimation angle decreases in an etendue limited optical system (fixed optical diameter, fixed emitter surface area), preserved output resulting from the ability to use an unfiltered source coupled with optimal fluorescent response from a carefully matched excitation source are key in that by allowing a single emitter to replace multiple same-sized emitters, the multiple optics necessary to collimate the multiple light sources may be replaced by a single optic of much greater diameter than each of the individual elements of the compound or multiple collimator array capable of much higher collimation or, at the same collimation angle, much higher optical efficiency in directing light into the desired area to be illuminated.

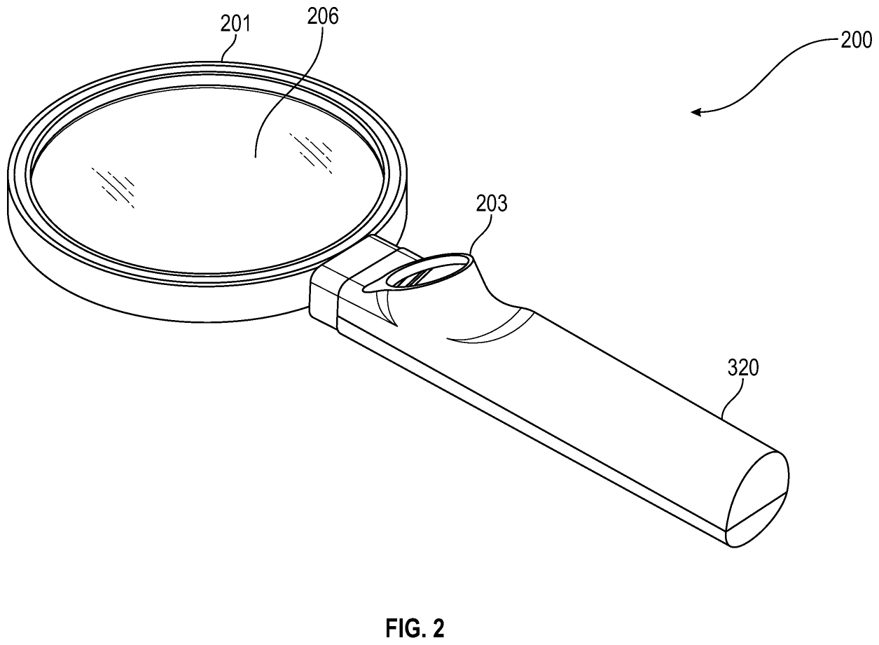

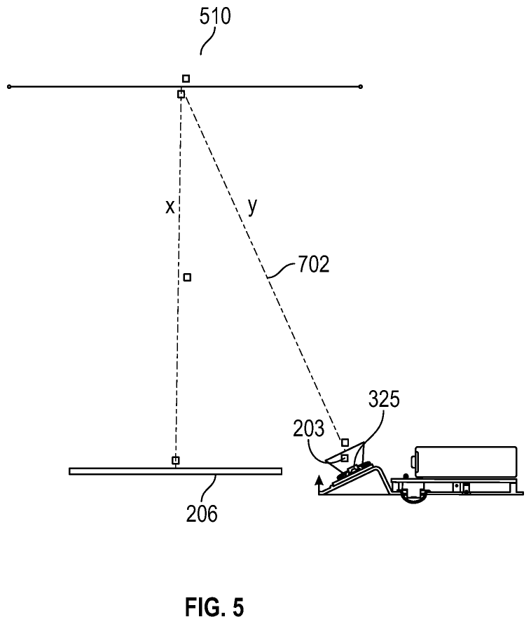

The emitted light is directed to skin or cutaneous surface of living tissue and penetrates to and including but not limited to subcutaneous epithelial tissue. Preferably the incidence of light is substantially normal to the tissue surface. Reference FIG. 5. As discussed, the device may be used for diagnosing epithelial neoplasia. It will be appreciated that the configuration of the Applicant's device as shown in FIGS. 2 and 3 facilitates this positioning of both the light emitter and detection viewer. Stated differently, both the incident (emitted) light and fluorescent light detector may be substantially normal to the subject target tissue. It will be appreciated that this is facilitated by adjusting the distance between the tissue surface 510 and the device 200 of FIG. 3.

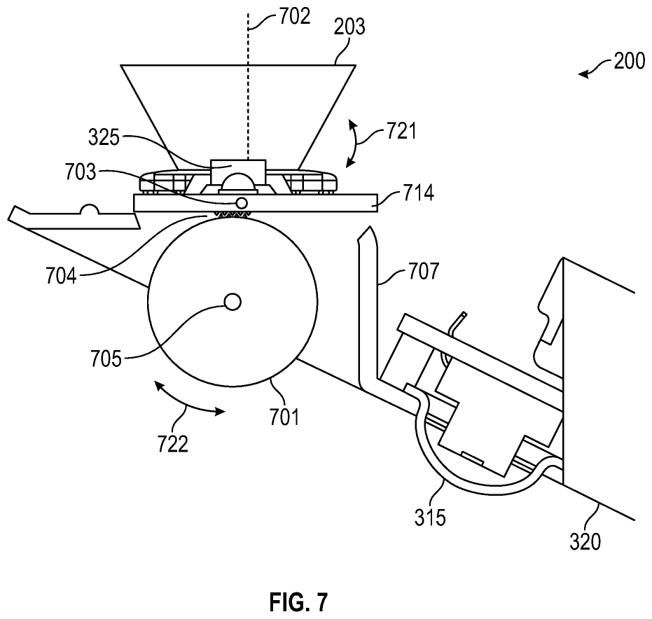

In an embodiment, the collimator or light emitter may pivot on the handle component 320 of the hand held device 200 illustrated in FIG. 3 to vary the angle of incidence to the surface of the living tissue. This pivoting may be controlled by the user. An example of this configuration is shown in FIG. 7. This embodiment utilizes a wheel 701 rotatable on an axle 705 within the handle component and controllable by the user and the diameter of the wheel engages with the pivotable light 325 or collimator 203 via a pivot 703 and complementary pivot wheel engagement components 704, e.g., gear teeth or similar. Rotation of the wheel 701 is shown by vector arrow 722. This movement causes movement of the LED 325, collimator 203 and axis 702 as shown by vector arrow 721. It will be appreciated that it may be advantageous to vary the axis 702 of emitted light with the distance or orientation of the hand held detector 200 positioned relative to the tissue surface. It will be appreciated that the wheel and connecting components may be adjacent to a heat sink 707. Also movement of the pivot wheel control 701 will cause the axis of the light emission 702 to move as shown by vector arrows 721 and 722.

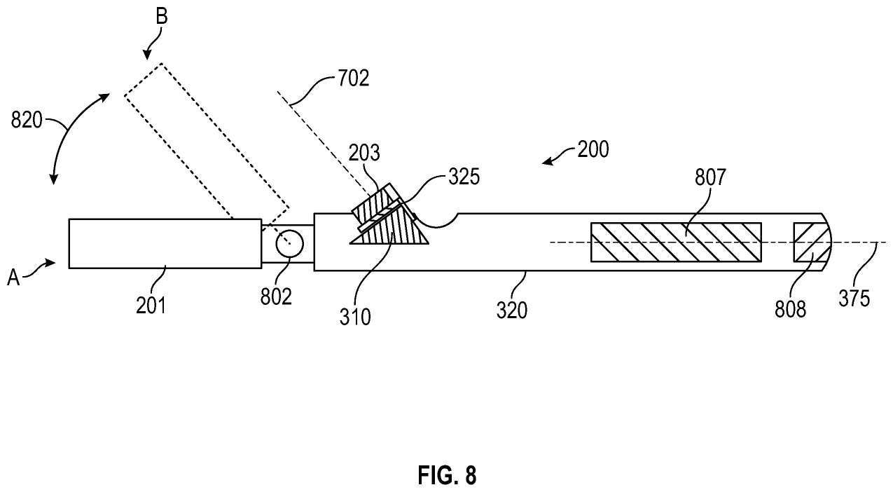

In an alternate embodiment illustrated in FIG. 8, the axis of the view port receptor 201 may be varied in relation to the axis 702 of the LED/collimator component. This can be accomplished by a pivot or hinged mechanism 802 connecting the view port receptor to the handle 320. Such an arrangement is illustrated in FIG. 8 wherein the receptor 201 with the low pass filter can pivot in relation to the axis 702 and attached handle 320 utilizing a pivot, hinge or similar mechanism 802. The receptor can move from position A to position B as shown by vector arrow 820. It will be appreciated that the position of the receptor can be varied and is not limited to the two illustrated positions. For example, the angle of the receptor may be greater than 180° to the handle axis 375. Also illustrated is the collimator 203, LED light source 325, heat sink 310. Also illustrated is an optional battery power source 807 and optional AC charging connection 808. The electrical connections among the components, e.g., LED and battery, are not shown.

As discussed above, the epithelial tissue contains a collagen matrix that auto fluoresces when exposed to the emitted light. The wavelength of this fluorescence may be approximately 550 nm. This relevant fluorescence may be described as being in the green-yellow range of 500 to 600 nm.

It will be appreciated that diseased tissue may not contain this collagen matrix. This tissue may be neoplastic and possibly cancerous. The absence of tissue fluorescence signatures is of particular interest, because spectral changes might reflect changes in metabolic activity and communication between the epithelium and the stroma, i.e., the stroma being supportive tissue of an epithelial organ, tumor, gonad, etc., consisting of connective tissues and blood vessels.

For example, the surface area of the tissue not exhibiting fluorescence, i.e., dark spots, may indicate neoplastic tissue growth.

An embodiment of the disclosure teaches viewing the illuminated tissue utilizing a hand held detector device 200 comprising an LED light. Referencing FIG. 2, the device comprises a handle 320 that may be comfortably held by the user with one hand. The handle may comprise protrusions or indentations or other structures to facilitate gripping the handle.

The handle may contain an energy source such as a battery, an activation switch, and LED emitter and collimator 203, e.g. wave guide optic. In other embodiments, the handle may include a charging port such as a micro USB port. See FIG. 8, 808.

Attached to the handle is a fluorescence light detector 201. The light detector contains a translucent long band pass filter 206. In one embodiment, such filter substantially blocks all visible wavelengths shorter than 500 nm and substantially passes all visible wavelengths longer than that value. Contrast the percent of light transmission within the blocking range 601 with the transmitted light wavelengths 603 as referenced in FIG. 6. Note also the steep vertical curve 602 illustrating the transition of wavelength passing through the filter 603 and wavelengths being blocked 601. Recall the range of relevant (induced) autofluorescence is between 500 nm and 600 nm. It will also be appreciated that the selected emitted light wavelength subject of this disclosure will be below 500 nm, its reflection off of target tissue thereby blocked by the long band pass filter and not interfering with the detection of induced fluorescence light as contrasted against non-fluorescing anomalous tissue.

It will be appreciated that the fluorescent light detector may be much small that illustrated in the figures of the disclosure. This will facilitate insertion of the device within narrow cavities of the body. The fluorescent light detector may be combined with a camera to facilitate viewing by the user. The camera may have a separately located display screen for the user.

In another embodiment, the device may include a battery charging port. The charging port may comprise a stand or attachment holding the device when not in use. The charging adaptor complementary to the charging stand could be in the handle. See for example FIG. 8. In other embodiments, the electrical supply for the LED component may be an AC electrical source. Other embodiments may include charging ports such as, but not limited to USB, micro USB or other common cell phone charge adaptors.

In another embodiment, the translucent long pass filter structure may also comprise an optical magnifier. This can allow the view of tissue surface markings or indicia that may be relevant to absence or weak fluorescence of a tissue area.

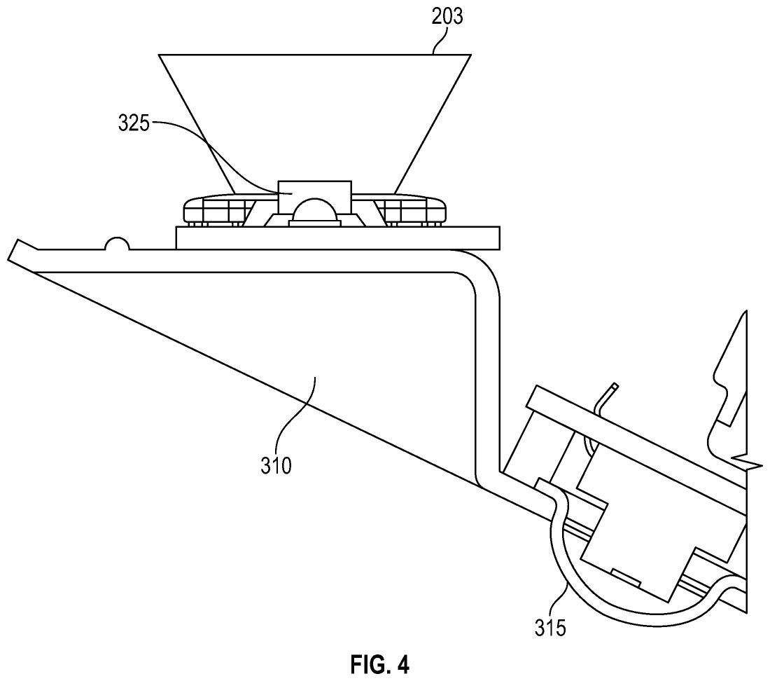

In another embodiment, the light emitter and collimator may be positioned on the frame of the translucent long pass filter structure. This configuration may allow the angle between the axis of light emission and axis of fluorescence observed with the device being diminished. This is referenced in the following discussion of FIGS. 4 and 5 below and particularly with regard to the angle designated “Y”.

Referencing FIG. 3, the device 200 illustrated in FIG. 2 is shown in a cut off perspective or cross section view. FIG. 3 illustrates the device handle 320, containing the location of a rechargeable battery 325 (power source), an internal LED driver 321, heat sink 310, activation switch 315 (which may be operated by the user's hand holding the device).

Referencing FIG. 4 and FIG. 5, it will be appreciated that the LED light 325, with emitted light guided by the collimator 203 (e.g., wave guide), is oriented to the area of tissue 510 also subject of viewing by the light detector 206. Angle Y of FIG. 5 illustrates the angle between the axis 702 of orientation of the collimated emitted light and the axis of view detector. It will be appreciated that in some embodiments the size of Y will be minimized.

It will be appreciated that the Applicant's disclosure includes but is not limited to the following advantages:
1. No costly dichroic filter required.
2. Because all of the LED emission band is within the excitation range and none extends beyond the viewing long band pass filter cutoff, no emitted light is lost due to a filtration requirement. This allows a single LED to provide the same level of tissue excitation as larger, more complex light systems used in prior art devices.
3. Because the Applicant's device uses a single emitter, a single, vastly more efficient collimator is used in place of the compound (triple) collimator by some prior art devices, resulting in a vastly higher percentage of emitted light being directed into the desired field. This also lowers the energy requirements.
4. All components reside within a single device rather than separate viewer, excitation emitter and charger. All of the components may be contained in a hand held system facilitating ease of examination.

Description of one embodiment of the disclosure:

A handheld device in which or to which is mounted an LED emitter of a specific output band, a beam-shaping collimating optic affixed to the LED, a heat sink to which the LED is mounted, an LED driver and battery charge management circuit, a charging port, and a low pass filtered viewport; such that emissions from the LED are directed by its collimator to a sample of human tissue in which auto-fluorescence is excited by the frequency of light output by the LED wherein the LED has a specified narrow emitted range of light wavelength, and which tissue may be viewed through the viewport filter such that reflected emissions from LED are either at a wavelength significantly different than the wavelength of the induced fluorescence or are blocked by said filter while fluorescent emissions from said excited tissues remain unfiltered. Emissions from the LED are in the blue light to ultraviolet range and within the excitation range of targeted biological tissues within the samples being examined, while the low pass viewport filter substantially blocks wavelengths emitted by the LED while passing substantially all wavelengths of the induced fluorescence light emitted by the excited tissues.

In another embodiment, the disclosure teaches an emission source within the excitation range of the fluorescence of the epithelial tissue but the excitation emission light curve does not intersect the fluorescence band of the excited tissue.

In yet another embodiment, the long pass filter has a cutoff, e.g., 500 nm as shown in FIG. 6, is at a longer wavelength than the longest wavelength emitted by the fluorescence excitation light source, e.g., LED light source, and the long band pass filter passes substantially all the desired longer wavelength fluorescent emission band. It will be appreciated that the long band filter cutoff 602 is substantially vertical.

In one embodiment, shown in FIG. 6, the long band pass filter cuts off at 500-505 nm at the half-height (half max) of the cutoff curve 602. It will be appreciated that the absolute cutoff (shorter wavelength) for the filter will be at a longer wavelength than the longest wavelength emitted by the light source and the long pass filter passes substantially all the desired fluorescent emission band. Therefore it is desirable that the filter have as vertical a cutoff 602 as possible.

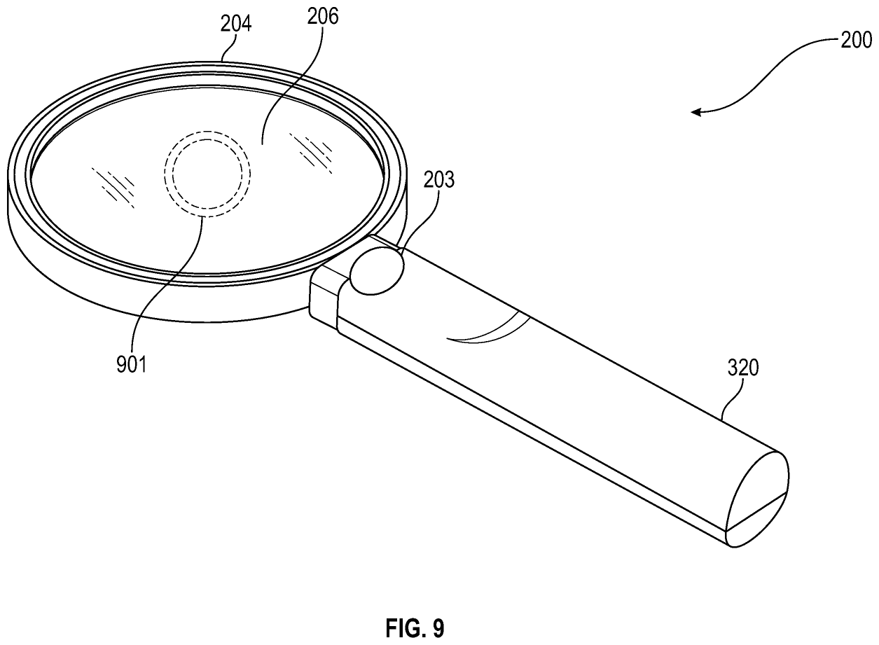

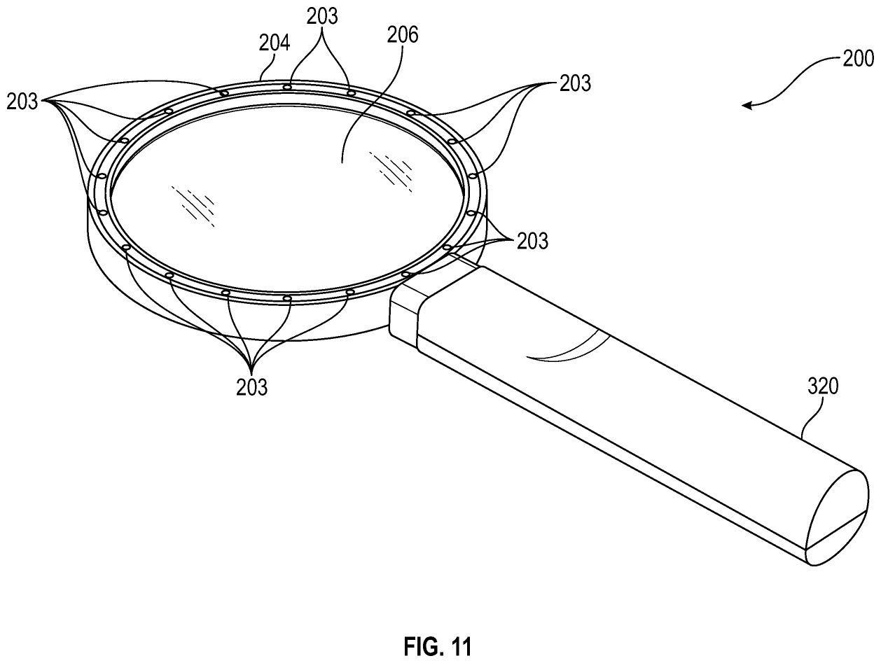

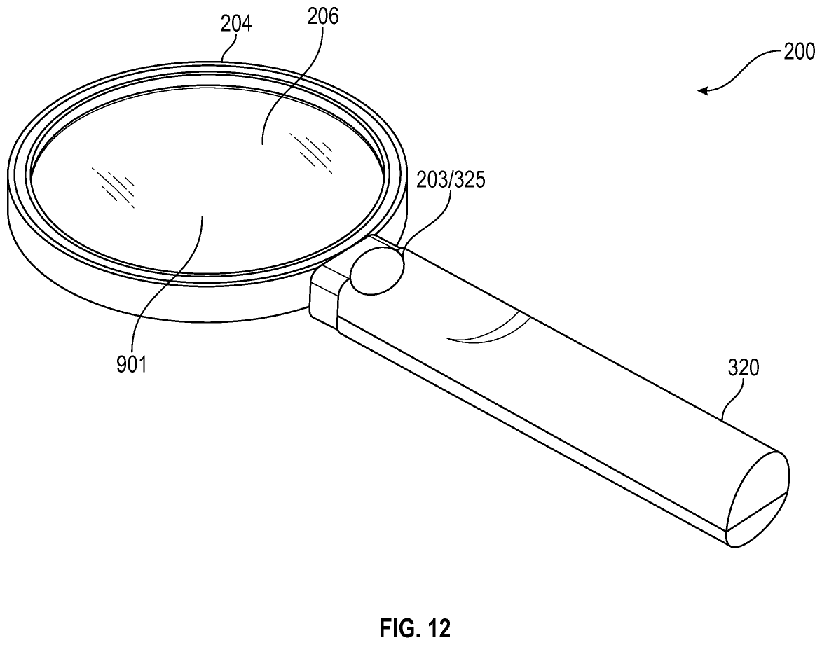

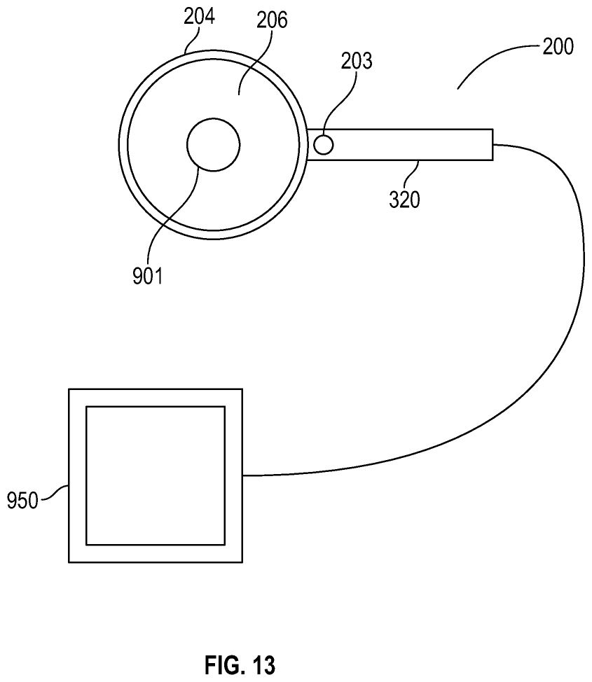

Further variations of the device and method include but are not limited to:
1. A device as described above in which all components are mounted within a single unitary housing.
2. A device as described above in which the LED and its power source and optics are mounted within one housing while the viewport is mounted separately.
3. A unitary device in which the LED and optic are mounted immediately adjacent to the viewport. See FIG. 12 showing the collimator 203 and underlying LED emitter 325 positioned adjacent to the frame 204 of the viewport filter 206.
4. A unitary device in which the LED and optic are mounted axially in the center of the viewport. It will be appreciated that this configuration will be similar to that of FIG. 9 wherein a camera 901 is positioned on the side of the viewport filter 206 opposite the target tissue (not shown).
5. A unitary device in which an array of LEDs of like wavelength and their collimating optic/s are mounted circumferentially around the perimeter of the viewport. See FIG. 11 illustrating multiple collimators 203 positioned on the border or frame 204 of the viewport filter 206. It will be appreciated that the LED emitters are positioned beneath each collimator.
6. A device in which the power supply and control electronics are mounted within the same housing as the LED, optic and heat sink.
7. A device in which the power supply and control electronics are mounted within a separate housing from the LED, optic and heat sink.
8. A device in which the viewport low pass filter is an absorptive media.
9. A device in which the viewport low pass filter is an interference (dichroic) filter.
10. A device in which the viewport low pass filter is designed to be mounted to a camera lens, including the camera lens of a cell phone. FIG. 9 illustrates a perspective view of this embodiment of the device 200 wherein a camera 901 capable of detecting the induced fluorescence within the tissue (not shown) is positioned on the opposite side of the viewport filter 206 from the tissue.
11. A device designed to be used with a camera and its attached filter as described above in which the LED excitation emitter and its optics are mounted separately.
12. The device of 11 wherein the images may be recorded.
13. A device 200 subject of this disclosure designed to be utilized with a camera 901 wherein the camera image can be displayed on a component 950 separate from the device. It will be appreciated that the camera of the device will receive images of the induced fluorescence within tissue resulting from the target tissue being illuminated with relatively short wavelength light 203/325, e.g., light of between 400 nm and 420 nm with a relatively narrow breadth of wavelengths such as the 0% range may be no greater than 50 nm from the wavelength peak and below the cutoff of the long pass filter comprising the viewport filter 206. See FIG. 13.
14. A device designed to be used with a camera and its attached filter as described above in which the LED excitation emitter and its optics are mounted in a unitary assembly with the viewport/lens filter. See FIG. 9.
15. A device as described in item 11 above in which the housing for the LED and optic includes a power supply and/or battery.
16. A device as described in item 11 above in which the housing for the LED and optic is powered externally, as with a cable to a cell phone battery or external power supply or phone charger.

Representative embodiments relate to providing a viewing device, such as filter glasses or cameras that capture the images from a sample to observe tissue fluorescence with a human eye.

In one respect, the invention involves a method for viewing an epithelial tissue sample that is being subjected to fluorescence inducing or exciting light waves. One or more wavelengths may between about 380 nm and 420 nm. The light waves maybe subject to collimation. The light waves are preferably directed substantially normal to the tissue surface. However, maintaining a clear observation aperture may require offset of the excitation emitter from normal. In such case, it is beneficial to mount the emitter at such angle as to triangulate center beam such that it intersects the target tissue substantially normal to center of the detector aperture when said filter is held at an optimal observation distance from the target tissue. It will be further appreciated that any reflected light from the excitation emitter will be blocked by the long pass filter of the detector structure. Reference is made to FIGS. 1 and 6.

Any induced fluorescence may be viewed or detected by a detector component. The detector component may include a long pass filter. The axial direction of the detector component is also substantially oriented to the tissue surface. The filtered image is viewed by a human eye. Further, viewing may include a camera that captures the fluorescent radiation from the sample and where the images produced from the camera may allow a human eye to discern between a normal tissue and a tissue containing epithelial neoplasia. It will also be appreciated to that software may be utilized to enhance the contrast between tissue surface exhibiting fluorescence and the absence of such fluorescence, thereby highlighting tissue perhaps subject of neoplasia or other condition. The contrast may be enhanced by software, thereby identifying areas meriting closer examination.

The epithelial neoplasia may include, but is not limited to, skin cancer, cervical cancer, colon cancer and oral cancer. Oral cancer may be defined as pharynx neoplasia, throat neoplasia, paranasal sinus neoplasia, nasal cavity neoplasia, larynx neoplasia, thyroid neoplasia, parathyroid neoplasia and/or salivary gland neoplasia.

This specification is to be construed as illustrative only and is for the purpose of teaching those skilled in the art the manner of carrying out the disclosure. It is to be understood that the forms of the disclosure herein shown and described are to be taken as the presently preferred embodiments. As already stated, various changes may be made in the shape, size and arrangement of components or adjustments made in the steps of the method without departing from the scope of this disclosure. For example, equivalent elements may be substituted for those illustrated and described herein and certain features of the disclosure maybe utilized independently of the use of other features, all as would be apparent to one skilled in the art after having the benefit of this description of the disclosure.

While specific embodiments have been illustrated and described, numerous modifications are possible without departing from the spirit of the disclosure, and the scope of protection is only limited by the scope of the accompanying claims.

What I claim is:

1. An epithelial neoplasia tissue detection device comprising:
(a) an electrical energy source in communication with an LED light wherein the LED light has a wavelength peak between 380 nm and 450 nm;
(b) a handle component containing the electrical energy source and LED light; and
(c) a translucent long pass filter affixed to the handle and structured to block light waves of substantially 500 nm or less.

2. The epithelial neoplasia tissue detection device of claim 1 further comprising the LED light having a narrow bandwidth.

3. The epithelial neoplasia tissue detection device of claim 2 wherein a half maximum of the bandwidth is not less than 90% or greater than 110% of the wavelength peak.

4. The epithelial neoplasia tissue detection device of claim 1 further comprising a 0% wavelength bandwidth not greater than 50 nm of the wavelength peak.

5. The epithelial neoplasia tissue detection device of claim 1 wherein the LED light is directed to a living tissue surface.

6. The epithelial neoplasia tissue detection device of claim 5 wherein the LED light is combined with a collimator to focus the light direction.

7. The epithelial neoplasia tissue detection device of claim 1 wherein the LED light induces fluorescence within the living tissue and the emitted fluorescence can be viewed through the long pass filter.

8. The epithelial neoplasia tissue detection device of claim 1 wherein the long pass filter is moveably adjustable on the handle.

9. The epithelial neoplasia tissue detection device of claim 8 wherein the long pass filter pivots on an end to the handle.

10. The epithelial neoplasia tissue detection device of claim 6 further comprising the collimated LED light has an axis of focus wherein the axis of focus can be moved in relation to the handle component.

11. The epithelial neoplasia tissue detection device further comprising a camera for receiving through the long pass filter induced fluorescence for separate display or recording.

12. A method for detecting fluorescence in epithelial tissue comprising the steps of:
(a) Irradiating the epithelial tissue from a light source wherein the wave length of the light source is substantially between 380 nm and 450 nm;
(b) Viewing fluorescence induced in the epithelial tissue through a long pass filter blocking wavelengths substantially 500 nm or less.

13. The method of claim 12 further comprising the light source and viewing component being a single device structured to be held in one hand.

14. The method of 12 displaying an image of the fluorescence transmitted through the long pass filter.

15. An epithelial neoplasia tissue detection device comprising:
(a) an electrical energy source in communication with an LED light wherein the LED light emits light having a wavelength less than 500 nm;
(b) a handle component containing the LED light; and
(c) a translucent long pass filter structured to receive fluorescent light induced by the LED light source wherein the translucent long pass filter is affixed to the handle to block light waves of substantially 500 nm or less.

16. The epithelial neoplasia tissue detection device of claim 15 wherein the handle includes a battery recharging component.

17. The device of claim 15 wherein at least a portion of the LED light is transmitted through a collimator.

18. The device of claim 17 further comprising a handle component that allows change in direction of an axial focus direction of the LED light transmitted through the collimator.

19. The device of claim 15 further comprising a handle component that allows change in the orientation of the translucent long pass filter in relation to the handle.

20. The device of claim 19 wherein the light emitted from the LED light source is not filtered.
